(19) United States
(12) Patent Application Publication
(10) Pub. No.: US 2005/0165275 A1
(43) Pub. Date: Jul. 28, 2005

(54) Inspection device insertion tube

(76) Inventors: Kenneth Von Felten, Cazenovia, NY (US); Raymond A. Lia, Auburn, NY (US)

Correspondence Address: WALL MARJAMA & BILINSKI, 101 SOUTH SALINA STREET, SUITE 400, SYRACUSE, NY, 13202, US

(21) Appl. No.: 10/763,131

(22) Filed: Jan. 22, 2004

(51) Int. Cl. A61B 1/00

(52) U.S. Cl. 600/140

(57) ABSTRACT

An insertion tube for an inspection device including a helically wound spiral tube, a first braided tube disposed about the helically wound spiral tube, a second braided tube disposed about the first braided tube, a first polymeric layer disposed between said first braided tube and said second braided tube and a polymeric layer coating the outer periphery of the second braided tube.

### FIELD OF THE INVENTION

The present invention relates generally to optical and video inspection devices and particularly to insertion tubes used, for example, in borescopes and endoscopes.

### BACKGROUND OF THE INVENTION

Inspection devices for the visual inspection of enclosed spaces generally fall into two categories, optical inspection devices and video inspection devices. Both optical inspection devices and video inspection devices typically include a viewer that is coupled to a flexible probe. The flexible probe includes an insertion tube and imaging optics. A distal end of the insertion tube may be configured to be articulated or fixed with respect to the remainder of the insertion tube, and typically includes imaging and illumination optics. The imaging optics for both types of inspection devices typically includes a lens system. The optical inspection device uses optical fibers to transmit the image to the viewer, while the video inspection device includes a digital image capturing device such as, for example, a CCD or CMOS-type imager. The captured image is then transmitted to the viewer as electrical signals. The insertion tube can include a central cavity in which the articulation controls for the proximal end and the wires or optical fibers for transmitting the image from the proximal end to the viewer are typically enclosed.

The above-noted inspection devices may be further divided into the operational categories of borescopes and endoscopes. Borescopes are inspection devices used to inspect confined areas such as the interior of conduits, the insides of structures and other hard to reach places, while endoscopes are medical devices that are used to inspect body cavities, such as the colon, intestines, esophagus, etc.

Both borescopes and endoscopes are operated by inserting the distal end of the insertion tube into the enclosed volume to be inspected, e.g. an automotive fuel tank or a body cavity, and directing the distal end to the target area that is of interest to the operator of the inspection device. The insertion portion should be as flexible as possible, and allow control by the remote operator, while still allowing illumination to be carried to the tip of the insertion portion and allowing image data to be transmitted therefrom. Conventional insertion tubes consist of a helically wound tube surrounded by a single braided wire sleeve with a polymeric coating applied to the outside of the braided wire sleeve. Typically, insertion tubes constructed according to this conventional design have exhibited at least two drawbacks. First, because insertion tubes of this construction commonly rely upon the polymeric outer layer to control the stiffness of the insertion tube, they develop a coil bias when coiled, such as, for example, when in storage. Insertion tubes that have developed a coil bias make use of the inspection device for certain activities more difficult. For example, borescopes are often used in an orientation where the insertion tube is required to hang straight down, one such example is in the inspection of nuclear fuel rods inside a reactor, a coil bias prevents the insertion tube from hanging vertically and thus, makes the inspection of the reactor difficult if not impossible. In another example, borescopes are often used to inspect the insides of pipes or conduits, an insertion tube that has developed an arcuate set may ride against the wall of the pipe or conduit thereby increasing the frictional forces that must be overcome in positioning the insertion tube within the pipe or conduit thereby making navigation of the insertion tube difficult for its operator. In another typical use, a bore scope is inserted into a large void through a small port. If the insertion tube has developed an arcuate set then inspecting certain regions of the void, such as straight across from the port, may be difficult if not impossible.

Second, insertion tubes of this type are typically constructed using a wire braided tube of constant braid angle, which translates into a constant stiffness. Thus, there is no provision for varying the stiffness of the insertion tube along its length. An insertion tube with a flexible distal end and a stiffer proximal end is desirable because the insertion tube will follow the distal end around corners and a more flexible distal end is better able to navigate around corners thus allowing greater inspection capability. A stiffer proximal end allows the operator to push the distal end of the insertion tube deeper into the cavity or void being inspected.

One proposed approach to providing a variable stiffness insertion tube includes providing an insertion tube having a spiral wound tube covered by a sheath, in which the spiral wound tube is coupled to a rotary member that is rotatable with respect to the insertion tube. Rotation of the rotary member changes the winding radius of the spiral wound tube, thereby changing the stiffness of the spiral wound tube. One drawback to this approach is that typically inspectors need the distal end less stiff than the proximal end and do not need to adjust the stiffness of the insertion tube. Another potential drawback to this approach is that it requires the operator to control the stiffness of the insertion tube. Furthermore, this approach requires additional mechanisms which may fail, for example changing the winding radius of the spiral tube may pinch fibers and/or cables disposed in the interior of the insertion tube.

Another proposed approach to providing a variable stiffness insertion tube includes providing an insertion tube having a composite helical tube formed by an inner helical tube and an outer helical tube wound coaxially with one another; the inner and outer helical tubes having two different winding radii. During use, the operator controls the overall stiffness of the insertion tube by varying the winding radius of the inner helical tube. Generally it is a design goal of industrial endoscope insertion tube to meet the end user's durability requirements with an insertion tube having the greatest possible inner diameter to outer diameter ratio. For a given outer diameter this provides the greatest internal volume which may be used for additional illumination or stronger articulation. Generally, this requires the a small thickness tube. For a given outer diameter, doubling the helical tube requires a greater insertion tube wall thickness which in turn reduces the available internal volume.

Yet another proposed approach to operationally varying the stiffness of an insertion tube includes providing an insertion tube having a spiral wound tube covered by a sheath. The stiffness of the insertion tube is changed by longitudinally compressing the spiral wound tube. The longitudinal compression of the spiral wound tube may be accomplished by a multitude of means including, for example, bending wires and tensioning wires. The proposed approach has the potential drawback that it requires the user to control the stiffness of the insertion tube. Furthermore, this approach requires additional mechanisms which may fail.

Still another proposed approach to operationally varying the stiffness of an insertion tube is to provide slip-on sheaths that fit over an exterior portion of the insertion tube, thereby increasing the stiffness of the insertion tube. The slip-on sheaths may be of different lengths and made from different materials than that of the insertion tube, thereby providing a range of potential stiffness increases for the insertion tube. One potential drawback of this approach is that if the opening into which the insertion tube is to be inserted is small, a slip-on sheath to provide the required stiffness may have too large a diameter to be effective thus rending the insertion tube useless for that application.

Yet still another proposed approach to providing a variable stiffness insertion tube includes dividing the insertion tube into a number of longitudinal segments. Each segment includes a helical coil formed from a memory metal, and the stiffness of the memory metal helical coil is controlled by heating the memory metal helical coil, which in turn causes a change in the geometry of the memory metal helical coil, and thereby changes its stiffness. The memory metal helical coil of each longitudinal segment is independently controllable, thereby allowing the operator to vary the longitudinal stiffness of the insertion tube. One potential drawback of this approach is that industrial endoscopes, or borescopes, are often required to operate in a wide temperature band. Therefore, a method that uses heat sensitive elements to control the stiffness of the insertion tube may suffer unexpected and/or uncontrollable changes in stiffness when subjected to widely varying operational temperatures.

Yet still another proposed approach to providing a variable stiffness insertion tube includes providing an insertion tube that includes a frame member, a tubular braid member disposed over the frame member and a outer cover over the tubular braid member. In this approach, the frame member is a shape memory member in the form of a helical tube, and the stiffness of the insertion tube is controlled by varying the stiffness of the helical tube. Variations in the stiffness of the helical tube may be accomplished by providing sections of different width and/or thickness. As noted with the previous approach, a number of potential problems arise when using heat sensitive stiffness elements in borescopes. Additionally, the varying the thickness and/or width of the bands of the helical coil effects the bend radius of the insertion tube, thereby limiting the functionality of the insertion tube.

Another proposed approach for controlling the flexibility of an insertion tube includes providing an insertion tube consisting of a helical tube, a braided tube covering the helical tube and a sheath covering the braided tube. The sheath is formed by applying a pliable material to the outside of the braided tube and forcing the sheath material through the interstices of the braided tube such that the sheath material protrudes through the openings in the helical tube and forms a plurality of protrusions that effective lock the helical tube, the braided tube and the sheath together. One potential drawback of this approach is that the manufacturing process of this approach requires a high amount of control over the amount of the sheath material that is forced through the braid. The difficulty in controlling the variations in the manufacturing process can lead to a high reject rates during the manufacture of the insertion tubes.

### SUMMARY OF THE INVENTION

It would thus be desirable in some instances to have an inspection device insertion tube that is stiffer toward the user, the proximal end and more flexible at the insertion or distal end, without experiencing the drawbacks associated with the approaches described above. It is also desirable to have an inspection device insertion tube that does not exhibit the drawbacks associated with an insertion tube that develops a biasing set while coiled for prolonged periods of time.

Therefore, and according to one illustrative embodiment of the present invention, there is provided an inspection device insertion tube. The inspection device insertion tube includes a helically wound spiral tube, a first braided tube disposed over at least a portion of the helically wound spiral tube, a second braided tube disposed over at least a portion of the first braided tube, a first polymeric layer disposed between said first braided tube and said second braided tube and a second polymeric layer coating the outer periphery of the second braided tube. As used herein the term tube refers to a member having a generally tubular or cylindrical configuration.

According to another embodiment of the present invention, an inspection device insertion tube includes a helically wound spiral tube and a first wire braid tube disposed over at least a portion of the helically wound spiral tube. The first wire braid tube has a first braid angle. As used herein the term “braid angle” is defined as the angle between the longitudinal axis of a braided tube and the wires or metallic fibers that are interwoven to form the braided tube. The inspection device insertion tube further includes a second wire braid tube disposed over at least a portion of the first wire braid tube. The second wire braid tube has a second braid angle that is different than the first braid angle. The inspection device insertion tube further includes a first polymeric layer disposed between the first wire braid tube and the second wire braid tube. A second polymeric coating covers the second wire braid tube.

In yet another embodiment of the present invention, an inspection device insertion tube includes a resilient helical coil member configured to allow a predetermined amount of elastic deformation. The inspection device insertion tube further includes a first resilient braided member. The first resilient braided member defines a first interior volume and has a polymeric coating disposed on the outer surface thereof. The resilient helical coil member is disposed within the first interior volume. A second resilient braided member defining a second interior volume is disposed such that the first resilient braided member is disposed within the second interior volume. A resilient coating disposed about the exterior of the second resilient braided member.

In yet another embodiment of the present invention a method is disclosed for making an inspection device insertion tube. The method includes the steps of providing a helical coil member and placing a first braid over at least a portion of the helical coil member, the first braid having a first braid angle. The method further includes the step of depositing a first polymeric layer on an outer peripheral surface of the first braid. A second braid is placed over at least a portion of the first braid, the second braid having a second braid angle, wherein the second braid angle is different from the first braid angle and a second polymeric layer is deposited on an outer peripheral surface of the second braid.

According to yet another embodiment of the present invention, an inspection device includes a viewer and an elongate flexible member having a proximal end and a distal end. The proximal end is coupled to the viewer, which may be an optical lens in a fiberscope device or a video display screen in a videoscope device. The elongate flexible member includes a resilient helical coil member configured to allow a predetermined amount of elastic deformation; a first resilient braided member having a polymeric coating disposed on the outer surface thereof, a second resilient braided member, and a resilient coating disposed about the exterior of said second resilient braided member. The first resilient braided member defines a first interior volume in which the resilient helical coil member is disposed. The second resilient braided member defines a second interior volume in which the first resilient braided member is disposed. The inspection device further includes an imaging module disposed at the distal end of the elongate flexible member. The imaging module includes imaging optics in communication with the viewer and a light source configured to illuminate a target area.

Additional features and advantages of the invention will be set forth in the Detailed Description which follows, and in part will be readily apparent to those skilled in the art from that description or recognized by practicing the invention as described herein, including the Detailed Description which follows, the claims, as well as the appended drawings.

It is to be understood that both the foregoing general description and the following detailed description are merely illustrative examples of the invention, and are intended to provide an overview or framework for understanding the nature and character of the invention as it is claimed. The accompanying drawings are included to provide a further understanding of the invention, and are incorporated in and constitute a part of this specification. The drawings illustrate various embodiments of the invention, and together with the description serve to explain the principles and operations of the invention.

### BRIEF DESCRIPTION OF THE DRAWINGS

FIG. 1 is an inspection device in which the present invention is embodied.

FIG. 1A is a cross-sectional side view of an insertion tube of the inspection device of FIG. 1;

FIG. 2 is an enlarged fragmentary cross sectional view of the insertion tube shown in FIG. 1A;

FIG. 2A is an enlarged fragmentary cross sectional view of the insertion tube shown in FIG. 1A;

FIG. 3A is an enlarged fragmentary view of a braided tube with a braid angle of 45 degrees used in one embodiment of the insertion tube of the present invention;

FIG. 3B is an enlarged fragmentary view of a braided tube having a braid angle of 30 degrees used in one embodiment of the insertion tube of the present invention; and

FIG. 4 is a perspective view of an insertion tube with a strains relief member according to one embodiment of the present invention.

### DETAILED DESCRIPTION OF THE PREFERRED EMBODIMENTS

Reference will now be made in detail to the present preferred embodiments of the invention, examples of which are illustrated in the accompanying drawings. Whenever possible, the same reference numerals will be used throughout the drawings to refer to the same or like parts for clarity.

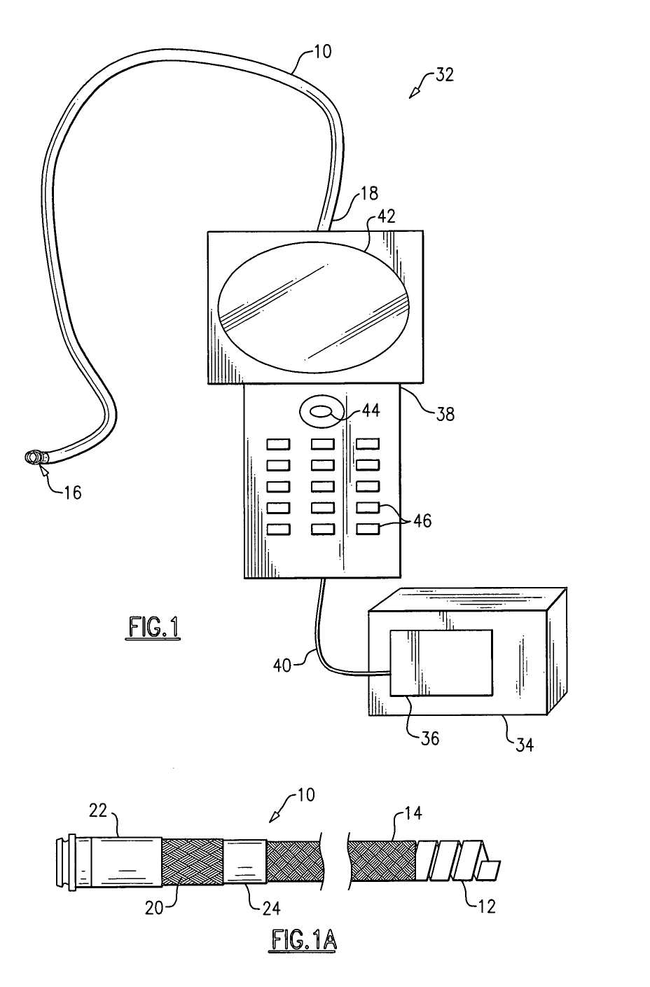

Referring to FIG. 1, a typical inspection device 32 (a borescope in the illustrative embodiment) according to the invention is illustrated, such as is sold by Everest VIT® of Flanders, N.J. Such a device could include, as shown in the illustrative embodiment, a portable shipping/operating case 34, that includes a power supply 36 for the device and a light source, such as a metal halide arc lamp (not shown). The shipping/operating case 34 is shown in operative communication with a handpiece 38 by means of a cable 40. The handpiece 38 can include, by way of example, an LCD monitor 42 (that displays images seen by the imaging device), a joystick control 44 (for articulating a distal end 16 of the inspection device 32), and a button set 46 (for accessing measurement, digital, and measurement controls associated with the imaging device 32). The handpiece 38 also is connected to an insertion tube 10, the insertion tube 10 terminating in a distal end 16. As used herein, the term “distal” shall mean “in the direction of the tip of the inspection device, furthest from the handpiece 38.” The insertion tube 10 can be sized according to the desired application, by varying a diameter and a length of the insertion tube 10. The interior of the insertion tube 10 (not shown) can include standard imager lines and communication/control means, such as fiber-optic cables and articulation wires.

In the above, the description was given for the case where the flexible tube for a borescope according to the present invention is applied to an electronic borescope (electronic type borescope). However, it is to be noted that the flexible tube of this invention may also be applied to a fiberscope (optical type borescope or endoscope).

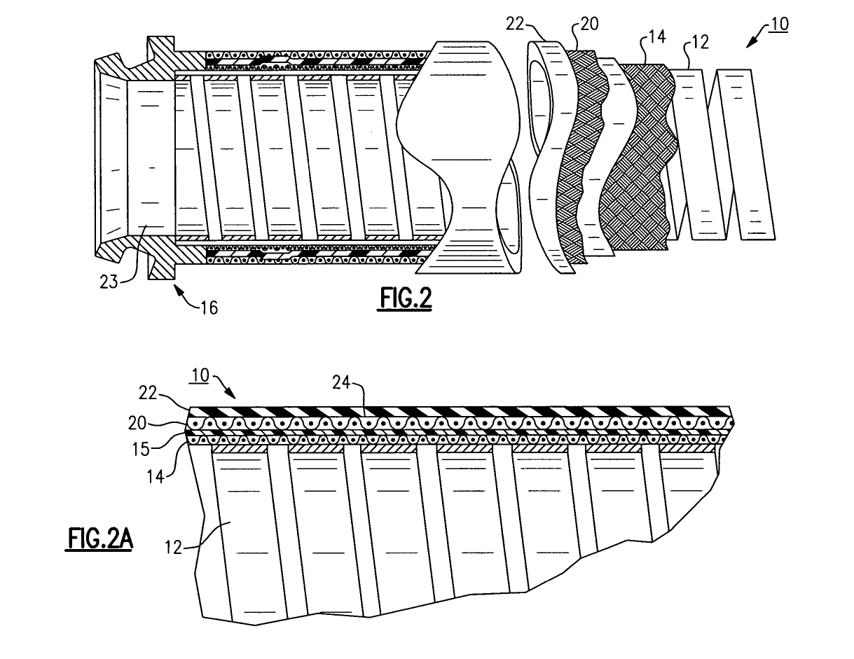

Referring to FIGS. 1a, 2 and 3 there is shown an insertion tube 10 made in accordance with a preferred embodiment of the present invention. The insertion tube 10 includes a flexible helical coil 12, also referred to herein as a monocoil. The flexible helical coil 12 is a resilient structure that is configured for elastic deformation into continuous curved forms. The flexible helical coil 12 may be, for example, a flexible spiral tube made from a thin section of stainless steel that is helically wound into a cylindrical tubular cross section. According to the present embodiment, the flexible helical coil 12 is made from stainless steel, although other structural materials may be easily substituted, such as, for example aluminum, titanium and plastics.

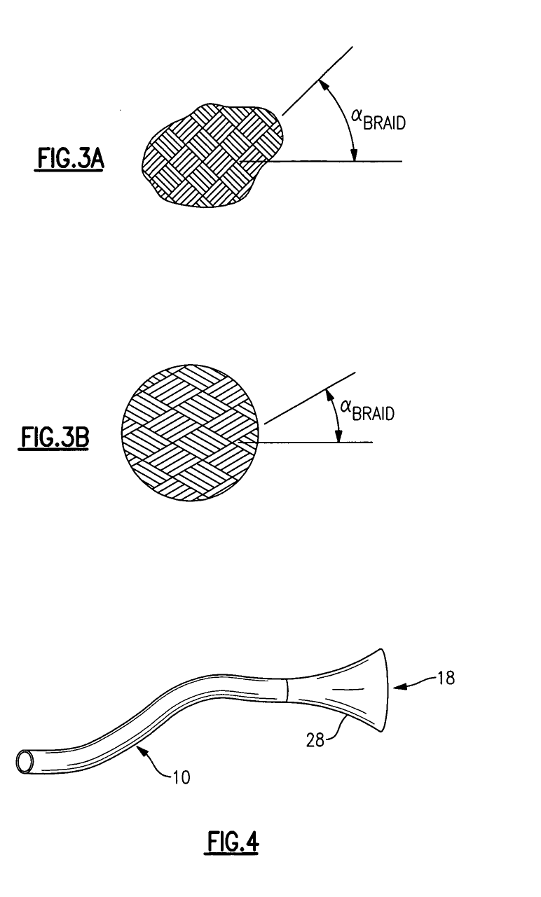

A first braided tube 14, which in one embodiment is a net-like braided structure, formed of interwoven metallic or other fibers, is placed in an overlaying relation onto the entirety of the length of the flexible helical coil 12. The first braided tube 14 in the illustrative embodiment is configured to provide the longitudinal stiffness of the insertion tube. In one embodiment, the first braided tube 14 is formed from a plurality of groups of wires, in which each group includes four wires. Each group of wires forms an angle with the longitudinal axis of the first braided tube 14, this angle being referred to as the braid angle, which may be better understood by referring to FIGS. 3A and 3B. Conventionally, the braid angle is kept constant along the entire length of the first braided tube and is typically about 45 degrees, however, the braid angle of the first braided tube 14 of the present invention may be constant or may vary along the length of the first braided tube 14. As will be appreciated by those skilled in the art, for a given wire diameter and material used to make a braided tube, the closer the braid angle to the longitudinal axis of the braided tube the stiffer the braided tube will be. Conversely, the further the braid angle is from the longitudinal axis of the braided tube, the more flexible the braided tube will be. Preferably, the braid angle at any point along the length of the first braided tube 14 is less than about 45 degrees. In one embodiment of the present invention, the braid angle of the first braided tube 14 is maintained at about 45 degrees for the entire length of the first braided tube 14. In an alternative embodiment, the braid angle of the first braided tube 14 varies along its length from about 15 degrees at the proximal end to about 45 degrees at the distal end. Varying the braid angle along the length of the first braided tube 14 in such a manner provides a more flexible region near the distal end of the insertion tube 10 and a stiffer region near the proximal end of the insertion tube 10. A more flexible distal end makes it easier to maneuver the distal end and its associated optics around obstacles, such as, for example, navigating sharp corners in a conduit. A stiffer proximal end makes it easier to push the insertion tube to overcome frictional forces or sticking and thereby advance the distal end and its associated optics deeper into the inspection area.

In yet another embodiment, the braid angle of the first braided tube 14 is about 30 degrees for the entire length of the first braided tube 14. FIG. 3A shows a section of a braided tube having a braid angle α_{BRAID} of about 45 degrees and FIG. 3B shows a section of a braided tube having a braid angle α_{BRAID} of about 30 degrees. The first braided tube 14 may be made from a stainless steel or other suitable material, such as aluminum, titanium or alloys thereof, plastics and polymers. In one embodiment, the first braided tube 14 is configured to be an electromagnetic interference inhibiting covering, such as, for example, by using stainless steel wire or other electromagnetic interference inhibiting material to form the first braided tube 14. According to the present embodiment, the first braided tube 14 is soldered or otherwise fixedly attached to the flexible helical coil 12 at respective distal and proximate ends 16, 18.

The insertion tube 10 in the illustrative embodiment further includes an intermediate polymeric layer 15 over the first braided tube 14. The intermediate polymeric layer impregnates the first braided tube 14 but does not penetrate to the flexible helical coil 12. The intermediate polymeric layer 15 may be, for example, a layer of black polyurethane 0.010 inch thick having a Shore 80 A durometer reading. The intermediate polymeric layer may be applied by extrusion, spraying, brushing or other conventional polymer application techniques. Alternatively, the intermediate polymeric layer may be a preformed sleeve or sheath that is slideably engageable with the first braided tube 14 or the first braided tube 14 may be wrapped with a polymer covering.

A second braided tube 20, which preferably is a net-like braided structure, formed of interwoven metallic or other fibers, is placed in an overlaying relation onto the entirety of the length of the first braided tube 14 such that the intermediate polymeric layer is disposed between the first braided tube 14 and the second braided tube 20. The braid angle of the second braided tube 20 is selected to complement the braid angle of the first braided tube 14, thereby providing a desired amount of stiffness as well as promoting the even flexing of the insertion tube 10. The braid angle of the second braided tube 20 is used to promote the even flexing of the insertion tube 10 while the braid angle of the first braided tube 14 is used to control the stiffness of the insertion tube 10. As will be appreciated by those skilled in the art, however, the functionality of the first and seconded braided tubes 14, 20 may be reversed such that the first braided tube 14 is configured to promote even flexing and the second braided tube 20 is configured to control the stiffness of the insertion tube 10. The braid angle of the second braided tube 20 may be either constant or vary along the length of the second braided tube 20. In one embodiment, the second braided tube 20 has a constant braid angle of about 45 degrees. The second braided tube 20 is preferably made from tungsten wire or other suitable material. In one embodiment, the second braided tube 20 is made up of 46 groups of NS-20 tungsten wire. Each of the 46 groups of NS-20 tungsten wire includes three wires. The second braided tube 20 is coupled to the distal and proximal ends 16, 18 of the flexible helical coil 12.

A thin polymeric layer 22 is then applied to the outer peripheral surface 24 of the second braided tube 20 and to the exterior portion of each end collar 23, 25 through conventional means such as, for example, spraying, painting, brushing, applying a preformed sleeve or sheath, or by wrapping. According to one embodiment, the polymer used is a two-part, low viscosity polyurethane dispersion which can be applied at room temperature and allowed to cure. Curing may take place at either room temperature or by placing the tubular assembly into an appropriately sized oven and heating, depending on the bonding requirements of the polymeric material used. Other suitable materials may also be used.

Collars 23, 25 are respectively attached to the distal and proximate ends 16, 18. According to one embodiment, each of the end collars 23, 25 are cylindrical stainless steel members having an appropriately sized interior cavity 26, including an annular shoulder 28 against which the ends of the tube sub-assembly are retained by soldering or adhesive bonding. The end collars 23, 25 are attached after the tube sub-assembly has been cut to length. The collar 23 attached to the distal end 16 is configured for engagement with an optical imaging bending section (not shown) while the collar 25 attached to the proximate end 18 is configured for engagement with a handset or display (not shown).

The insertion tube 10 may further include a strain relief member 28 as shown in FIG. 4. The strain relief member 28 is a polymeric member that fits over the proximate end 18 of the insertion tube 10 and is coupled to the insertion tube 10. The strain relief member has a variable cross section that gradually decreases from a stiff section adjacent to the proximate end 18 of the insertion tube 10. The strain relief member 28 is configured for engagement with a handset or display (not shown) and serves to prevent a stress concentration where the proximate end 18 is coupled to the handset or display, thereby reducing the stress levels in the insertion tube 10.

It will be apparent to those skilled in the art that various modifications and variations can be made to the present invention without departing from the spirit and scope of the invention. Thus it is intended that the present invention cover the modifications and variations of this invention provided they come within the scope of the appended claims and their equivalents.

1. An insertion tube for an inspection device comprising:
a helically wound spiral tube;
a first braided tube disposed at least partially over said helically wound spiral tube;
a second braided tube disposed at least partially over said first braided tube;
a first polymeric layer disposed between said first braided tube and said second braided tube, and
a second polymeric layer coating the outer periphery of said second braided tube.

2. The insertion tube for an inspection device of claim 1 wherein said first braided tube includes a wire braid having a braid angle of about 45 degrees relative to the longitudinal axis of said first braided tube.

3. The insertion tube for an inspection device of claim 1 wherein said first braided tube includes a wire braid having a braid angle of less than about 45 degrees relative to the longitudinal axis of said first braided tube.

4. The insertion tube for an inspection device of claim 3 wherein said second braided tube includes a wire braid having a braid angle of about 45 degrees relative to the longitudinal axis of said second braided tube.

5. The insertion tube for an inspection device of claim 1 wherein said second braided tube includes a wire braid having a braid angle of greater than about 45 degrees relative to the longitudinal axis of said second braided tube.

6. The insertion tube for an inspection device of claim 1 wherein said first braided tube includes a wire braid having a braid angle that varies along the length of said wire braid.

7. The insertion tube for an inspection device of claim 6 wherein said second braided tube includes a wire braid having a braid angle of about 45 degrees relative to the longitudinal axis of said second braided tube.

8. The insertion tube for an inspection device of claim 6 wherein said second braided tube includes a wire braid having a braid angle greater than about 45 degrees relative to the longitudinal axis of said second braided tube.

9. The insertion tube for an inspection device of claim 1 wherein said first braided tube includes a plurality of stainless steel wires.

10. An insertion tube for an inspection device comprising:
a helically wound spiral tube;
a first wire braided tube disposed about said helically wound spiral tube, said first wire braided tube having a first braid angle;
a second wire braided tube disposed about said first wire braided tube, said second wire braided tube having a second braid angle;
a first polymeric layer disposed between said first wire braided tube and said second wire braided tube, and
a polymeric coating covering said second wire braid tube;
wherein said first braid angle is less than said second braid angle.

11. The insertion tube for an inspection device of claim 10 wherein said first wire braid tube includes at least a first plurality of wire groups.

12. The insertion tube for an inspection device of claim 11, wherein said second wire braid tube includes a second plurality of wire groups.

13. The insertion tube for an inspection device of claim 12, wherein each wire group of said first plurality of wire groups includes at least three wires.

14. The insertion tube for an inspection device of claim 12, wherein each wire group of said second plurality of wire groups includes at least four wires.

15. The insertion tube for an inspection device of claim 10, wherein said second braid angle is about 45 degrees relative to the longitudinal axis of said second braided tube.

16. The insertion tube for an inspection device of claim 10, wherein said second braid angle is greater than about 45 degrees relative to the longitudinal axis of said second braided tube.

17. The insertion tube for an inspection device of claim 10, wherein said first wire braid tube includes a plurality of stainless steel wires.

18. The insertion tube for an inspection device of claim 10, wherein said second wire braid tube includes a plurality of tungsten wires.

19. The insertion tube for an inspection device of claim 10, wherein said polymeric coating includes a polyurethane having about a Shore 85A durometer.

20. The insertion tube for an inspection device of claim 10, wherein said first braid angle varies from about 15 degrees to about 45 degrees over the length of said first wire braid tube.

21. The insertion tube for an inspection device of claim 10, wherein said first polymeric layer includes a polyurethane having about a Shore 80 A durometer.

22. An insertion tube for an inspection device comprising:
a resilient helical coil member configured to allow a predetermined amount of elastic deformation;
a first resilient braided member, said first resilient braided member defining a first interior volume, wherein said resilient helical coil member is disposed within said first interior volume, said first resilient braided member having a polymeric coating disposed on the outer surface thereof;
a second resilient braided member, said second resilient braided member defining a second interior volume, wherein said first resilient braided member is disposed within said second interior volume; and
a resilient coating disposed about the exterior of said second resilient braided member.

23. A method for making a insertion tube for an inspection device comprising the steps of:
providing a helical coil member;
placing a first braid over at least a portion of the helical coil member, the first braid having a first braid angle;
depositing a first polymeric layer on an outer peripheral surface of the first braid;
placing a second braid over at least a portion of the first braid, the second braid having a second braid angle; and
depositing a second polymeric layer on an outer peripheral surface of the second braid;
wherein the first braid angle is different from the second braid angle.

24. An inspection device comprising:
a viewer;
an elongate flexible member having a proximate end and a distal end, the proximate end coupled to the viewer, the elongate flexible member including:
a resilient helical coil member configured to allow a predetermined amount of elastic deformation;
a first resilient braided member, the first resilient braided member defining a first interior volume, wherein the resilient helical coil member is disposed within the first interior volume, the first resilient braided member having a polymeric coating disposed on the outer surface thereof;
a second resilient braided member, the second resilient braided member defining a second interior volume, wherein the first resilient braided member is disposed within the second interior volume; and
a resilient coating disposed about the exterior of the second resilient braided member; and
an imaging optical system disposed at the distal end of the elongate flexible member, the imagining optical system in communication with the viewer and further including a light source configured to illuminate a target area.

25. The inspection device of claim 24, wherein the viewer includes a display.

26. The inspection device of claim 25, wherein the imaging module includes a CCD imager.
